(19) United States
(12) Patent Application Publication
(10) Pub. No.: US 2022/0315594 A1
(43) Pub. Date: Oct. 6, 2022

(54) METHOD OF SYNTHESIS OF COMPOUND FOR DUAL INHIBITION OF JAK2 AND BET

(71) Applicant: OHM ONCOLOGY, Austin, TX (US)

(72) Inventors: Ram Shankar Upadhayaya, Journalist Colony (IN); Murali Krishna Ukkalam, Journalist Colony (IN); K Narasimha Sarma, Journalist Colony (IN)

(73) Assignee: OHM ONCOLOGY, Austin, TX (US)

(21) Appl. No.: 17/634,215

(22) PCT Filed: Aug. 9, 2020
(86) PCT No.: PCT/US2020/045555

§ 371 Date: Feb. 9, 2022

Related U.S. Application Data
(60) Provisional application No. 62/885,190, filed on Aug. 9, 2019.

(51) Int. Cl. C07D 487/04; A61K 31/437; A61K 35/00

(52) U.S. Cl. CPC C07D 487/04; A61K 31/437; A61K 35/00

(57) ABSTRACT

Embodiments of the invention include quinoline compounds and methods of synthesis. Ohm-581 has demonstrated dual inhibition of JAK2 and BET and acts as a therapeutic agent for micosis fungoides (MF) and other hematologic malignancies. Embodiments also include an efficient process for the preparation of Ohm-581 and pharmaceutically acceptable salts. The process is suited for large-scale production of quinoline compounds including Ohm-581.

### FIELD OF THE INVENTION

The invention relates to quinoline compounds and methods of synthesis, and more specifically, it relates to a method of preparation of N-(3-(2-((4-(4-(dimethylamino)piperidin-1-yl)-3-fluorophenyl)amino)-7H-pyrrolo[2,3-d]pyrimidin-7-yl)phenyl)-2-methylpropane-2-sulfonamide (Ohm-581) and pharmaceutically acceptable acid salts.

### BACKGROUND

Histone acetylation and deacetylation are the processes by which the lysine residues within the N-terminal tail protruding from the histone core of the nucleosome are acetylated and deacetylated as part of gene regulation. Histone acetylation and deacetylation are essential to gene regulation and chromatin organization in eukaryotic cells. Acetylation refers to the process where an acetyl functional group is transferred from one molecule (in this case, Acetyl-Coenzyme A) to another. Deacetylation refers to the reverse reaction where an acetyl group is removed from a molecule. These reactions are typically catalyzed by enzymes with “histone acetyltransferase” (HAT) or “histone deacetylase” (HDAC) activity.

Histone acetylation controls gene expression by recruiting protein complexes that bind directly to acetylated lysine via bromodomains. Bromodomain (BRD)-containing proteins are essential for the recognition of acetylated lysine (KAc) residues of histones during transcriptional activation. Bromodomain (BRD) can recognize a class of histone acetylation lysine (KAc) conserved protein domain and bind to acetylated lysine cause chromatin remodeling-associated protein enrichment factors and transcription factors, gene transcription in specific sites, alteration of the activity of RNA polymerase II and transcription regulation of gene expression. The bromodomain and extra terminal domain (BET) proteins, include Brd2, Brd3, Brd4, and BrdT, each of which contains two bromodomains in tandem that can independently bind to acetylated lysines.

BRDs regulate the transcription of various oncogenes, such as c-Myc and Bcl-2. Thus, BRDs have emerged as promising drug targets for a number of disease pathways that are characterized by changes in the epigenetic cell signature. To date, only a few structurally diverse BRD inhibitors have been reported, all of which specifically target the KAc recognition sites of the bromodomain and extra terminal (BET) family of proteins (BRD2, BRD3, BRD4, and BRDT), each containing two tandem BRDs.

BET inhibitors are believed to be useful in the treatment of diseases or conditions related to systemic or tissue inflammation, inflammatory responses to infection or hypoxia, cellular activation and proliferation, lipid metabolism, fibrosis, and the prevention and treatment of viral infections. Interfering with BET protein interactions via bromodomain inhibition results in modulation of transcriptional programs that are often associated with diseases characterized by dysregulation of cell cycle control, inflammatory cytokine expression, viral transcription, hematopoietic differentiation, insulin transcription, and adipogenesis.

Recent studies demonstrate that BET-inhibitors exert a broad spectrum of desirable biological effects such as anticancer and anti-inflammatory properties. Recently, it was discovered that the BRD of BETs interact with diverse kinase inhibitors. Janus kinase 2 (JAK2) is a non-receptor tyrosine kinase which catalyzes the transfer of a phosphate group from a nucleoside triphosphate donor, such as ATP, to tyrosine residues in proteins. JAK2 is involved in various processes such as cell growth, development, differentiation or histone modifications. Accordingly, gain-of-function mutations in JAK2 have been implicated in cancer cell growth and progression, formation of metastasis, and tumor neovascularization. Dual targeting of bromodomains and kinases, such as BRD4 and JAK2, offers a promising new strategy to treat conditions mediated by bromodomain activity and tyrosine kinase activity.

Ohm-581, referred to herein as the compound of “Formula I,” has demonstrated dual inhibition of JAK2 and BET. However, this compound is difficult to synthesize and conventional methods have been ineffective at large scale preparation. An object of the present invention is to solve the problems associated with the preparation of Ohm-581 and to provide an efficient process for the preparation of pharmaceutically acceptable acid salts. It is a further object of the present invention to provide a process that is cost effective, safe, and convenient in large-scale production of Ohm-581, analogues and salts.

### SUMMARY OF THE INVENTION

The following summary is provided to facilitate an understanding of some of the innovative features unique to the disclosed embodiment and is not intended to be a full description. A full appreciation of the various aspects of the embodiments disclosed herein can be gained by taking into consideration the entire specification, claims and abstract as a whole.

Embodiments include a compound having a structure according to the following formula:

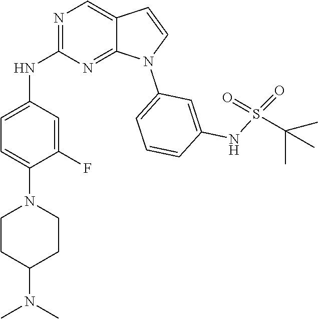 [SMILES: CN(C)C1CCN(c2ccc(Nc3ncc4ccn(-c5cccc(NS(=O)(=O)C(C)(C)C)c5)c4n3)cc2F)CC1]

a stereoisomer, or a salt thereof. Embodiments also include a process for the preparation of the compound including steps of (a) oxidizing a sulfone intermediate with an oxidizing agent in a solvent, (b) condensing the solvent in presence of a metal halide and a catalyst, (c) isolating one or more reaction products from the solvent at one or more pH levels and (d) identifying the reaction product as Ohm-581.

### Definitions

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can be in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

As applicable, the terms “about” or “generally”, as used herein in the specification and appended claims, and unless otherwise indicated, means a margin of +/−20%. Also, as applicable, the term “substantially” as used herein in the specification and appended claims, unless otherwise indicated, means a margin of +/−10%. It is to be appreciated that not all uses of the above terms are quantifiable such that the referenced ranges can be applied.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

As used herein, the term “hydrate” refers to a crystal form with either a stoichiometric or non-stoichiometric amount of water is incorporated into the crystal structure.

The term “alkenyl” as used herein refers to an unsaturated straight or branched hydrocarbon having at least one carbon-carbon double bond, such as a straight or branched group of 2-8 carbon atoms, referred to herein as (C2-C8)alkenyl. Exemplary alkenyl groups include, but are not limited to, vinyl, allyl, butenyl, pentenyl, hexenyl, butadienyl, pentadienyl, hexadienyl, 2-ethylhexenyl, 2-propyl-2-butenyl, and 4-(2-methyl-3-butene)-pentenyl.

The term “alkoxy” as used herein refers to an alkyl group attached to an oxygen (—O-alkyl-). “Alkoxy” groups also include an alkenyl group attached to an oxygen (“alkenyloxy”) or an alkynyl group attached to an oxygen (“alkynyloxy”) groups. Exemplary alkoxy groups include, but are not limited to, groups with an alkyl, alkenyl or alkynyl group of 1-8 carbon atoms, referred to herein as (C1-C8)alkoxy. Exemplary alkoxy groups include, but are not limited to methoxy and ethoxy.

The term “alkyl” as used herein refers to a saturated straight or branched hydrocarbon, such as a straight or branched group of 1-8 carbon atoms, referred to herein as (C1-C8)alkyl. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, and octyl.

The term “alkynyl” as used herein refers to an unsaturated straight or branched hydrocarbon having at least one carbon-carbon triple bond, such as a straight or branched group of 2-8 carbon atoms, referred to herein as (C2-C8)alkynyl. Exemplary alkynyl groups include, but are not limited to, ethynyl, propynyl, butynyl, pentynyl, hexynyl, methylpropynyl, 4-methyl-1-butynyl, 4-propyl-2-pentynyl, and 4-butyl-2-hexynyl.

The term “amide” as used herein refers to the form —NRaC(O)(Rb)— or —C(O)NRbRc, wherein Ra, Rb and Rc are each independently selected from alkyl, alkenyl, alkynyl, aryl, arylalkyl, cycloalkyl, haloalkyl, heteroaryl, heterocyclyl, and hydrogen. The amide can be attached to another group through the carbon, the nitrogen, Rb, or Rc. The amide also may be cyclic, for example Rb and Rc, may be joined to form a 3- to 8-membered ring, such as 5- or 6-membered ring. The term “amide” encompasses groups such as sulfonamide, urea, ureido, carbamate, carbamic acid, and cyclic versions thereof. The term “amide” also encompasses an amide group attached to a carboxy group, e.g., -amide-COOH or salts such as -amide-COONa, an amino group attached to a carboxy group (e.g., -amino-COOH or salts such as -amino-COONa).

The term “amine” or “amino” as used herein refers to the form —NRdRe or —N(Rd)Re—, where Rd and Re are independently selected from alkyl, alkenyl, alkynyl, aryl, arylalkyl, carbamate, cycloalkyl, haloalkyl, heteroaryl, heterocyclyl, and hydrogen. The amino can be attached to the parent molecular group through the nitrogen. The amino also may be cyclic, for example any two of Rd and Re may be joined together or with the N to form a 3- to 12-membered ring (e.g., morpholino or piperidinyl). The term amino also includes the corresponding quaternary ammonium salt of any amino group. Exemplary amino groups include alkylamino groups, wherein at least one of Rd or Re is an alkyl group. In some embodiments Rd and Re each may be optionally substituted with hydroxyl, halogen, alkoxy, ester, or amino.

The term “aryl” as used herein refers to a mono-, bi-, or other multi-carbocyclic, aromatic ring system. The aryl group can optionally be fused to one or more rings selected from aryls, cycloalkyls, and heterocyclyls. The aryl groups of this present disclosure can be substituted with groups selected from alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide, and thioketone. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. Exemplary aryl groups also include, but are not limited to a monocyclic aromatic ring system, wherein the ring comprises 6 carbon atoms, referred to herein as “(C6)aryl.”

The term “arylalkyl” as used herein refers to an alkyl group having at least one aryl substituent (e.g., -aryl-alkyl-). Exemplary arylalkyl groups include, but are not limited to, arylalkyls having a monocyclic aromatic ring system, wherein the ring comprises 6 carbon atoms, referred to herein as “(C6)arylalkyl.”

A “bromodomain” refers to an approximately 110 amino acid protein domain that recognizes acetylated lysine residues, such as those on the N-terminal tails of histones. As the “readers” of lysine acetylation, bromodomains are responsible for transducing the signal carried by acetylated lysine residues and translating it into various normal or abnormal phenotypes.

The term “carbamate” as used herein refers to the form —Rgoc(O)N(Rh)—, —Rgoc(O)N(Rh)Ri—, or —oc(O)NRhRi, wherein Rg, Rh and Ri are each independently selected from alkyl, alkenyl, alkynyl, aryl, arylalkyl, cycloalkyl, haloalkyl, heteroaryl, heterocyclyl, and hydrogen. Exemplary carbamates include, but are not limited to, arylcarbamates or heteroaryl carbamates (e.g., wherein at least one of Rg, Rh and Ri are independently selected from aryl or heteroaryl, such as pyridine, pyridazine, pyrimidine, and pyrazine).

The term “carboxy” as used herein refers to —COON or its corresponding carboxylate salts (e.g., —COONa). The term carboxy also includes “carboxycarbonyl,” e.g. a carboxy group attached to a carbonyl group, e.g., —C(O)—COOH or salts, such as —C(O)—COONa.

The term “cyano” as used herein refers to —CN.

The term “cycloalkoxy” as used herein refers to a cycloalkyl group attached to an oxygen.

The term “cycloalkyl” as used herein refers to a saturated or unsaturated cyclic, bicyclic, or bridged bicyclic hydrocarbon group of 3-12 carbons, or 3-8 carbons, referred to herein as “(C3-C8)cycloalkyl,” derived from a cycloalkane. Exemplary cycloalkyl groups include, but are not limited to, cyclohexanes, cyclohexenes, cyclopentanes, and cyclopentenes. Cycloalkyl groups may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Cycloalkyl groups can be fused to other cycloalkyl saturated or unsaturated, aryl, or heterocyclyl groups.

The term “dicarboxylic acid” as used herein refers to a group containing at least two carboxylic acid groups such as saturated and unsaturated hydrocarbon dicarboxylic acids and salts thereof. Exemplary dicarboxylic acids include alkyl dicarboxylic acids. Dicarboxylic acids may be substituted with alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydrogen, hydroxyl, ketone, nitro, phosphate, sulfide, sulfinyl, sulfonyl, sulfonic acid, sulfonamide and thioketone. Dicarboxylic acids include, but are not limited to succinic acid, glutaric acid, adipic acid, suberic acid, sebacic acid, azelaic acid, maleic acid, phthalic acid, aspartic acid, glutamic acid, malonic acid, fumaric acid, (+)/(−)-malic acid, (+)/(−) tartaric acid, isophthalic acid, and terephthalic acid. Dicarboxylic acids further include carboxylic acid derivatives thereof, such as anhydrides, imides, hydrazides (for example, succinic anhydride and succinimide).

The term “ester” refers to the structure —C(O)O—, —C(O)O—Rj-, —RkC(O)O—Rj-, or —RkC(O)O—, where O is not bound to hydrogen, and Rj and Rk can independently be selected from alkoxy, aryloxy, alkyl, alkenyl, alkynyl, amide, amino, aryl, arylalkyl, cycloalkyl, ether, haloalkyl, heteroaryl, and heterocyclyl. Rk can be a hydrogen, but Rj cannot be hydrogen. The ester may be cyclic, for example the carbon atom and Rj, the oxygen atom and Rk, or Rj and Rk may be joined to form a 3- to 12-membered ring. Exemplary esters include, but are not limited to, alkyl esters wherein at least one of Rj or Rk is alkyl, such as —O—C(O)-alkyl, —C(O)—O-alkyl-, and -alkyl-C(O)—O-alkyl-. Exemplary esters also include aryl or heteoraryl esters, e.g. wherein at least one of Rj or Rk is a heteroaryl group such as pyridine, pyridazine, pyrimidine and pyrazine, such as a nicotinate ester. Exemplary esters also include reverse esters having the structure —RkC(O)O—, where the oxygen is bound to the parent molecule. Exemplary reverse esters include succinate, D-argininate, L-argininate, L-lysinate and D-lysinate. Esters also include carboxylic acid anhydrides and acid halides.

The terms “halo” or “halogen” as used herein refer to F, CI, Br, or I.

The term “haloalkyl” as used herein refers to an alkyl group substituted with one or more halogen atoms. “Haloalkyls” also encompass alkenyl or alkynyl groups substituted with one or more halogen atoms.

The term “heteroaryl” as used herein refers to a mono-, bi-, or multi-cyclic, aromatic ring system containing one or more heteroatoms, for example 1-3 heteroatoms, such as nitrogen, oxygen, and sulfur. Heteroaryls can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heteroaryls can also be fused to non-aromatic rings. Illustrative examples of heteroaryl groups include, but are not limited to, pyridinyl, pyridazinyl, pyrimidyl, pyrazyl, triazinyl, pyrrolyl, pyrazolyl, imidazolyl, (1,2,3)- and (1,2,4)-triazolyl, pyrazinyl, pyrimidilyl, tetrazolyl, furyl, thienyl, isoxazolyl, thiazolyl, furyl, phenyl, isoxazolyl, and oxazolyl. Exemplary heteroaryl groups include, but are not limited to, a monocyclic aromatic ring, wherein the ring comprises 2-5 carbon atoms and 1-3 heteroatoms, referred to herein as “(C2-C5)heteroaryl.”

The terms “heterocycle,” “heterocyclyl,” or “heterocyclic” as used herein refer to a saturated or unsaturated 3-, 4-, 5-, 6- or 7-membered ring containing one, two, or three heteroatoms independently selected from nitrogen, oxygen, and sulfur. Heterocycles can be aromatic (heteroaryls) or non-aromatic. Heterocycles can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, nitro, phosphate, sulfide, sulfinyl, sulfonyl, sulfonic acid, sulfonamide and thioketone. Heterocycles also include bicyclic, tricyclic, and tetracyclic groups in which any of the above heterocyclic rings is fused to one or two rings independently selected from aryls, cycloalkyls, and heterocycles. Exemplary heterocycles include acridinyl, benzimidazolyl, benzofuryl, benzothiazolyl, benzothienyl, benzoxazolyl, biotinyl, cinnolinyl, dihydrofuryl, dihydroindolyl, dihydropyranyl, dihydrothienyl, dithiazolyl, furyl, homopiperidinyl, imidazolidinyl, imidazolinyl, imidazolyl, indolyl, isoquinolyl, isothiazolidinyl, isothiazolyl, isoxazolidinyl, isoxazolyl, morpholinyl, oxadiazolyl, oxazolidinyl, oxazolyl, piperazinyl, piperidinyl, pyranyl, pyrazolidinyl, pyrazinyl, pyrazolyl, pyrazolinyl, pyridazinyl, pyridyl, pyrimidinyl, pyrimidyl, pyrrolidinyl, pyrrolidin- 2-onyl, pyrrolinyl, pyrrolyl, quinolinyl, quinoxaloyl, tetrahydrofuryl, tetrahydroisoquinolyl, tetrahydropyranyl, tetrahydroquinolyl, tetrazolyl, thiadiazolyl, thiazolidinyl, thiazolyl, thienyl, thiomorpholinyl, thiopyranyl, and triazolyl.

The terms “hydroxy” and “hydroxyl” as used herein refer to —OH.

The term “hydroxyalkyl” as used herein refers to a hydroxy attached to an alkyl group.

The term “hydroxyaryl” as used herein refers to a hydroxy attached to an aryl group.

The term “ketone” as used herein refers to the structure —C(O)—Rn (such as acetyl, —C(O)CH3) or —Rn-C(O)—Ro-. The ketone can be attached to another group through Rn or Ro. Rn or Ro can be alkyl, alkenyl, alkynyl, cycloalkyl, heterocyclyl or aryl, or Rn or Ro can be joined to form a 3- to 12-membered ring.

The term “monoester” as used herein refers to an analogue of a dicarboxylic acid wherein one of the carboxylic acids is functionalized as an ester and the other carboxylic acid is a free carboxylic acid or salt of a carboxylic acid. Examples of monoesters include, but are not limited to, to monoesters of succinic acid, glutaric acid, adipic acid, suberic acid, sebacic acid, azelaic acid, oxalic and maleic acid.

The term “N-protecting group” refers to groups intended to protect an amino group against undesirable reactions during synthetic procedures. Commonly used N-protecting groups are disclosed in Greene, “Protective Groups in Organic Synthesis,”4th Edition (John Wiley & Sons, Hoboken, N.J., 2006), which is incorporated herein by reference. N-protecting groups include acyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, a-chlorobutyryl, benzoyl, 4-chlorobenzoyl, 4-bromobenzoyl, 4-nitrobenzoyl, and chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-n itro-4, 5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxy carbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, alkaryl groups such as benzyl, triphenylmethyl, benzyloxymethyl, and the like and silyl groups such as trimethylsilyl, and the like. Preferred N-protecting groups are formyl, acetyl, benzoyl, pivaloyl, t-butylacetyl, alanyl, phenylsulfonyl, benzyl, t-butyloxycarbonyl (Boc), and benzyloxycarbonyl (Cbz).

The term “phenyl” as used herein refers to a 6-membered carbocyclic aromatic ring. The phenyl group can also be fused to a cyclohexane or cyclopentane ring. Phenyl can be substituted with one or more substituents including alkoxy, aryloxy, alkyl, alkenyl, alkynyl, amide, amino, aryl, arylalkyl, carbamate, carboxy, cyano, cycloalkyl, ester, ether, formyl, halogen, haloalkyl, heteroaryl, heterocyclyl, hydroxyl, ketone, phosphate, sulfide, sulfinyl, sulfonyl, sulfonic acid, sulfonamide and thioketone.

The term “thioalkyl” as used herein refers to an alkyl group attached to a sulfur (—S-alkyl-).

The term “acetylation” or in IUPAC nomenclature “ethanoylation” refers to a reaction that introduces an acetyl functional group into a chemical compound. In contrast, deacetylation refers to the removal of an acetyl group.

“Alkyl,” “alkenyl,” “alkynyl”, “alkoxy”, “amino” and “amide” groups can be optionally substituted with or interrupted by or branched with at least one group selected from alkoxy, aryloxy, alkyl, alkenyl, alkynyl, amide, amino, aryl, arylalkyl, carbamate, carbonyl, carboxy, cyano, cycloalkyl, ester, ether, formyl, halogen, haloalkyl, heteroaryl, heterocyclyl, hydroxyl, ketone, phosphate, sulfide, sulfinyl, sulfonyl, sulfonic acid, sulfonamide, thioketone, ureido and N. The substituents may be branched to form a substituted or unsubstituted heterocycle or cycloalkyl.

As used herein, a suitable substitution on an optionally substituted substituent refers to a group that does not nullify the synthetic or pharmaceutical utility of the compounds of the present disclosure or the intermediates useful for preparing them. Examples of suitable substitutions include, but are not limited to: C1-8 alkyl, alkenyl or alkynyl, C1-6 aryl, C7-5 heteroaryl; C3-7 cycloalkyl; C1-8 alkoxy; C6 aryloxy; —CN; —OH; oxo; halo, carboxy; amino, such as —NH(C1-8 alkyl), —N(C1-8alkyl)2, —NH((C6)aryl), or —N((C6)aryl)2; formyl; ketones, such as —CO(C1-8 alkyl), —CO((C6aryl) esters, such as —CO2(C1-8 alkyl) and —CO2 (C6aryl). One of skill in art can readily choose a suitable substitution based on the stability and pharmacological and synthetic activity of the compound of the present disclosure.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutically acceptable composition” as used herein refers to a composition comprising at least one compound as disclosed herein formulated together with one or more pharmaceutically acceptable carriers.

The term “pharmaceutically acceptable prodrugs” as used herein represents those prodrugs of the compounds of the present disclosure that are, within the scope of sound medical judgment, suitable for use in contact with the tissues of humans and lower animals without undue toxicity, irritation, allergic response, commensurate with a reasonable benefit/risk ratio, and effective for their intended use, as well as the zwitterionic forms, where possible, of the compounds of the present disclosure. A discussion is provided in Higuchi et al., “Prodrugs as Novel Delivery Systems,” ACS Symposium Series, Vol. 14, and in Roche, E. B., ed. Bioreversible Carriers in Drug Design, American Pharmaceutical Association and Pergamon Press, 1987, both of which are incorporated herein by reference.

The term “pharmaceutically acceptable salt(s)” refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfate, citrate, matate, acetate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions, that are acidic in nature are capable of forming base salts with various pharmacologically acceptable cations. Examples of such salts include alkali metal or alkaline earth metal salts and, particularly, calcium, magnesium, sodium, lithium, zinc, potassium, and iron salts.

The “two theta” or “2Θ value” refers to a value determined by X-ray diffraction. The incident ray and reflected ray make the angle theta with a crystal plane. Reflections from planes set at theta angle with respect to the incident beam generates a reflected beam at an angle 2-theta from the incident beam.

The compounds of the disclosure may contain one or more chiral centers and/or double bonds and, therefore, exist as stereoisomers, such as geometric isomers, enantiomers or diastereomers. The term “stereoisomers” when used herein consist of all geometric isomers, enantiomers or diastereomers. These compounds may be designated by the symbols “R” or “S,” depending on the configuration of substituents around the stereogenic carbon atom. The present disclosure encompasses various stereoisomers of these compounds and mixtures thereof. Stereoisomers include enantiomers and diastereomers. Mixtures of enantiomers or diastereomers may be designated “(±)” in nomenclature, but the skilled artisan will recognize that a structure may denote a chiral center implicitly.

Individual stereoisomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, or (3) direct separation of the mixture of optical enantiomers on chiral chromatographic columns. Stereoisomeric mixtures can also be resolved into their component stereoisomers by well-known methods, such as chiral-phase gas chromatography, chiral-phase high performance liquid chromatography, crystallizing the compound as a chiral salt complex, or crystallizing the compound in a chiral solvent. Stereoisomers can also be obtained from stereomerically-pure intermediates, reagents, and catalysts by well-known asymmetric synthetic methods.

Geometric isomers can also exist in the compounds of the present disclosure. The present disclosure encompasses the various geometric isomers and mixtures thereof resulting from the arrangement of substituents around a carbon-carbon double bond or arrangement of substituents around a carbocyclic ring. Substituents around a carbon-carbon double bond are designated as being in the “Z” or “E” configuration wherein the terms “Z” and “E” are used in accordance with IUPAC standards. Unless otherwise specified, structures depicting double bonds encompass both the E and Z isomers.

Substituents around a carbon-carbon double bond alternatively can be referred to as “cis” or “trans,” where “cis” represents substituents on the same side of the double bond and “trans” represents substituents on opposite sides of the double bond. The arrangements of substituents around a carbocyclic ring are designated as “cis” or “trans.” The term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

The compounds disclosed herein may exist as tautomers and both tautomeric forms are intended to be encompassed by the scope of the present disclosure, even though only one tautomeric structure is depicted.

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

### DESCRIPTION OF PREFERRED EMBODIMENTS

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the structure particularly pointed out in the written description and claims hereof.

Bromodomain and extra terminal domain (BET) proteins include Brd2, Brd3, Brd4, and BrdT, each of which contain two bromodomains in tandem that can independently bind to acetylated lysines. BET inhibitors are a class of drugs that reversibly bind the bromodomains of Bromodomain and Extra-Terminal motif (BET) proteins BRD2, BRD3, BRD4, and BRDT, and prevent protein-protein interaction between BET proteins and acetylated histones and transcription factors.

BET inhibitors have many potential therapeutic uses including the treatment of various cancers. BET inhibitors may also be efficacious in atherosclerosis and associated conditions because of anti-inflammatory effects as well as ability to increase transcription of Apo A-I, the major constituent of HDL. BET inhibitors may also be useful in the prevention and treatment of conditions associated with ischemia-reperfusion injury such as myocardial infarction, stroke and acute coronary syndromes.

OHM-581 is a dual inhibitor of JAK2 and BET that has potential as a therapeutic agent for micosis fungoides (MF) and other hematologic malignancies. In vitro biochemical assays show OHM-581 to inhibit BRD4 BD1 and BD2 as well as JAK2. OHM-581 displays significant anti-proliferative activity against multiple liquid cancer cell lines. Consistent with its mechanism of action, OHM-581 robustly down-regulates cMYC expression and JAK2 signaling.

In one embodiment, the invention is directed to the compound OHM-581, also referred to as “Formula I.”

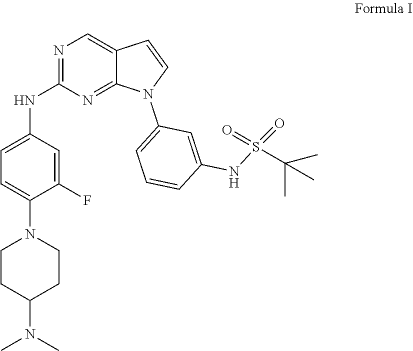 [SMILES: CN(C)C1CCN(c2ccc(Nc3ncc4ccn(-c5cccc(NS(=O)(=O)C(C)(C)C)c5)c4n3)cc2F)CC1]

The compound can also be described as of N-(3-(2((4-(4-(dimethylamino)piperidin-1-yI)-3-fluorophenyl)amino)-7H-pyrrolo[2,3-d]pyrimidin-7-yl)phenyl)-2-methylpropane-2-sulfonamide.

It will be appreciated that the invention covers compounds of Formula I, analogues and salts thereof. In one embodiment, the invention relates to compounds of Formula I in the form of a free base. In another embodiment, the invention relates to compounds of Formula I or a pharmaceutically acceptable salt thereof.

Because of their potential use in medicine, salts of the compound of Formula I are may be preferred as pharmaceutically acceptable salts. Suitable pharmaceutically acceptable salts can include acid addition salts. A pharmaceutically acceptable salt can be readily prepared by using a desired acid or base as appropriate. The resultant salt can precipitate from solution and be collected by filtration or recovered by evaporation of the solvent. The compound can exist as a stereoisomer, tautomer, pharmaceutical acceptable salt, or hydrate thereof:

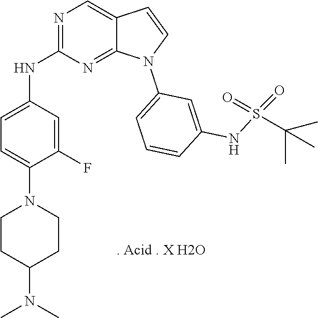 [SMILES: CN(C)C1CCN(c2ccc(Nc3ncc4ccn(-c5cccc(NS(=O)(=O)C(C)(C)C)c5)c4n3)cc2F)CC1]

### Utility and Administration

The compounds described herein are useful in the methods of the invention and, while not bound by theory, are believed to exert their desirable effects through their ability to reversibly bind the bromodomains of Bromodomain and Extra-Terminal motif (BET) proteins. The compounds described herein can also be used for the treatment of certain conditions in oncology such as myeloproliferative neopplasm (P vera, ET, post-MPN MDS/sAML), de novo and secondary MDS/AML, premalignant breast disease, CMML, acute and chronic GvHD, HSCT applications, gliomas/glioblastoma, Hodgkin lymphoma, diffuse large B-cell lymphoma, head and neck carcinomas, triple-negative breast cancer, prostate cancer (as one illustrative but not all inclusive example, androgen-independent prostate cancer), hypereosinophilic syndrome/primary eosinophilic disorders, T-cell leukemia/lymphoma including peripheral T-cell lymphomas, blastic plasmacytoid dendritic cell neoplasm, Chronic Lymphocytic Leukemia/Small Lymphocytic Lymphoma, Hemophagocytic Lymphohistiocytosis, multiple myeloma, Chuvash polycythemia, Atypical Chronic Myeloid Leukemia, BCR-ABL1 Negative, Chronic Neutrophilic Leukemia, Hodgkin Lymphoma and Primary Mediastinal Large B-cell Lymphoma, pretreatment of MF patients prior to allogeneic HSCT.

The compounds described herein can also be used for treatment of various autoimmune conditions including rheumatoid arthritis, psoriasis, psoriatic arthritis, ulcerative colitis, Crohn's disease and other forms of inflammatory bowel disease, noninfective uveitis and scleritis, various forms of spondyloarthritis including ankylosing spondylitis, reactive arthritis, enteropathic arthritis, juvenile enthesitis-related arthritis, non-radiographical axial spondyloarthritis, and undifferentiated spondyloarthritis, Sjogren's syndrome, chronic synovitis, atopic dermatitis, vitiligo, alopecia areata and alopecia universalis, frontal fibrosing alopecia, pemphigus, multiple sclerosis, scleroderma, lupus, dermatomyositis, juvenile arthritis of various forms such as juvenile rheumatoid arthritis.

Other potential uses include treatment of fibrotic diseases of multiple types including idiopathic pulmonary fibrosis and others, Bronchiolitis Obliterans Syndrome (BOS) After Allogeneic Hematopoietic Cell Transplantation (HCT), OHM-581 as a therapy to effect the numbers and functional activities of Tumor Infiltrating Myeloid Cells and Tumor Infiltrating Lymphocytes to reprogram tumor cells with resistance to cancer immunotherapies, HIV, treatment of cancer cachexia, lichen planus, lichen planoplaris, thalassemia major (to reduce spleen size), recalcitrant palmoplantar pustulosis, amyopathic dermatomyositis-associated interstitial lung disease, sarcoidosis, asthma, allergy-induced airway inflammation, familial Mediterranean fever, pyoderma gangrenosum, STING-associated vasculopathy with onset in infancy (SAVI), prevention of transplant rejection (as one example, prevention of chronic renal allograft rejection).

### Pharmaceutical Compositions

For use as treatment of human and animal subjects, the compounds of the invention can be formulated as pharmaceutical or veterinary compositions. Depending on the subject to be treated, the mode of administration, and the type of treatment desired (e.g., prevention, prophylaxis, or therapy) the compounds are formulated in ways consonant with these parameters. A summary of such techniques is found in Remington: The Science and Practice of Pharmacy, 21″ Edition, Lippincott Williams & Wilkins, (2005); and Encyclopedia of Pharmaceutical Technology, eds. J. Swarbrick and J.C. Boylan, 1988-1999, Marcel Dekker, New York, each of which is incorporated herein by reference.

The compounds described herein may be present in amounts totaling 1-95% by weight of the total weight of the composition. The composition may be provided in a dosage form that is suitable for intraarticular, oral, parenteral (e.g., intravenous, intramuscular), rectal, cutaneous, subcutaneous, topical., transdermal, sublingual., nasal, vaginal, intravesicular, intraurcthral, intrathecal, epidural, aural, or ocular administration, or by injection, inhalation, or direct contact with the nasal, genitourinary, gastrointestinal, reproductive or oral mucosa. Thus, the pharmaceutical composition may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, preparations suitable for iontophoretic delivery, or aerosols. The compositions may be formulated according to conventional pharmaceutical practice.

### Synthesis

The reaction scheme, steps and examples are intended to illustrate the synthesis of a representative number of compounds. Accordingly, the Examples are intended to illustrate but not to limit the invention. Additional compounds not specifically exemplified may be synthesized using conventional methods in combination with the methods described herein.

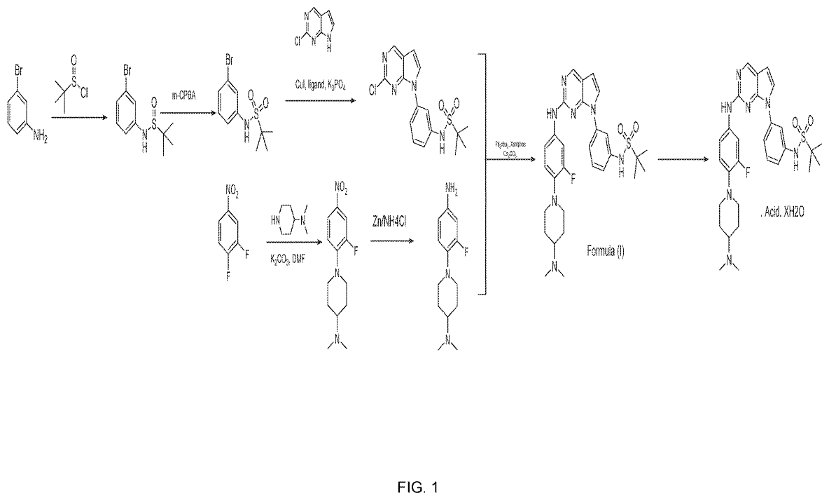

Embodiments include a process for the synthesis of the compound of Formula I and pharmaceutically acceptable acids, salts and organic, inorganic (ionic and covalent) salts and complexes thereof, substantially free of impurity. The process includes a step of oxidation of sulfone by an oxidizing agent in a solvent. FIG. 1 depicts the reaction scheme which is further detailed below. The process comprises steps of:
a) Oxidizing sulfone intermediate with an oxidizing agent in a solvent,
b) Condensation in the presence of a metal halide, catalyst at various pressures,
c) Isolation using high purity techniques at different pH levels,
d) Isolation and polymorph study of the Formula I compound, and
e) Treating the Formula I compound with different acids and organic/inorganic ionic complexes.

### Step I

Preparation of N-(3-bromophenyl)-2-methylpropane-2-sulfinamide

The compound of Formula I can be prepared by the procedure depicted in FIG. 1. The procedure can be divided into several steps. Step I begins with 3-Bromo Aniline and pyridine as described below.

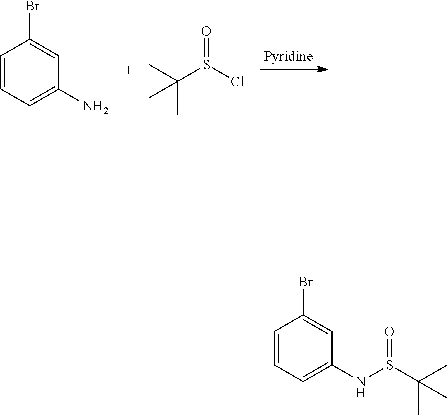

3-Bromo Aniline (100.0 g, 581.4 mmol) and pyridine (300 mL) were charged into a round bottom flask and stirred at room temperature. Tert-Butyl sulfuryl chloride (79.6 mL, 639.48 mmol) was added drop wise under nitrogen atmosphere at 0-5° C. over a period of 60-120 minutes. The reaction mass was gradually warmed to room temperature (25-30° C.) and stirred at this temperature for 3-4 hours. The reaction was monitored by thin layer chromatography (TLC). After completion of the reaction, the reaction mass was cooled to 0-5° C. and quenched by the addition of water (1000 mL) below 5° C. It was then warmed to room temperature and stirred at this temperature for 1 hour. The solid was collected by filtration, washed with water (500 mL), and suction dried for 1 hour. After suck drying for about an hour, the wet material was taken in hexane (300 mL) and heated to 50-55° C. for 3-4 h. The suspension was cooled to 25-30° C., the solid separated was filtered, washed with hexane (100 mL) and the material was dried for 10-15 h at 50-55° C. to get the product as off white solid.

The method described as Step I produced the following results:

H1-NMR Values: 7.187(s, 1H), 7.149-7.096(d, 2H), 6.918-6.945(m, 1H), 1.327(s, 9H)

Mass (ESI): m/z 275.8/276.6[m+H]+

Purity by HPLC: NLT 98.0%

Yield: 90-93%

### Step II

Preparation of 1-(2-fluoro-4-nitrophenyl)-N,N-dimethylpiperidin-4-amine

Step II begins with 4-Dimethyl amino piperidine as described below.

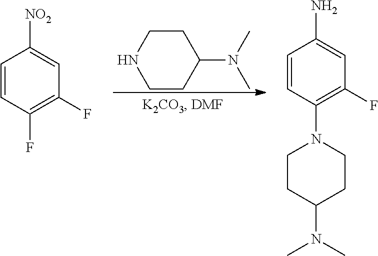

4-Dimethyl amino piperidine (100.0 g, 779.9 mmol) and DMF (500 mL) were combined in a round bottom flask, cooled to 0-5° C. and then stirred for 10-15 min. To this solution, potassium carbonate was added (323.36 g, 2339.6 mmol) lot wise at 0-5° C. The temperature was slowly raised to room temperature. Charged 1,2-di fluoro-4-nitrobenzene (123.76 g, 777.9 mmol) as added drop wise at 25-30° C. and the solution was stirred at room temperature for 2-3 hours. Chilled water (1000 mL) was added to the reaction mass and stirred for 1 hour, filtered and washed with water (1000 mL). The pale yellow solid was suction dried for 1-2 hours and used as such for next reaction.

The method described as Step II produced the following results:

H1-NMR Values: 7.953-7.953(dd, 1H), 7.874-7.912(dd, 1H), 6.886-6.931(t, 1H), 3.745-3.776(d, 2H), 2.866-2.927(t, 2H), 2.322-2.368(s, 7H), 1.936-1.967(d, 2H), 1.627-1.722(m, 2H)

Mass (ESI): m/z 268/270[m+H]+

Purity by HPLC: NLT 97.0%

Yield: 95-98%

### Step III

Preparation of 1-(4-amino-2-fluorophenyl)-N,N-dimethylpiperidin-4-amine

Step III begins with the nitro compound produced in Step II.

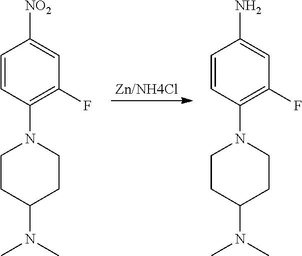 [SMILES: CN(C)C1CCN(c2ccc([N+](=O)[O-])cc2F)CC1 ; CN(C)C1CCN(c2ccc(N)cc2F)CC1]

The nitro compound (100.0 g, 374.1 mmol) was dissolved in water (1000 mL) and ammonium chloride (100.0 g, 1872.6 mmol) at room temperature then stirred for 15 minutes. Zn dust (122.0 g, 1868.3 mmol) was added and the temperature was slowly raised to 75-80° C. The solution was stirred for 2 hours. After completion of the reaction, aqueous ammonia (150 mL) was slowly added to reaction mass while maintaining the temperature below 30° C. (to control exothermicity). The reaction mass was filtered through a celite bed, washed with water (500 mL) and DCM (200 mL). The aqueous layer was washed with DCM (2×200 mL). Combined organic layers were completely distilled out under vacuum below 45° C. Charged n-heptane (mL) was added to the crude mass and stirred for 1 hour. The brown solid was filtered, washed with n-hexane (100 mL) suction dried and then dried at 55° C. for 10 hours.

The method described as Step Ill produced the following results:

H1-NMR Values: 6.792-6.809(t, 1H), 6.378-6.443(t, 2H), 3.525(br, 2H), 3.323-3.349(d, 2H), 2.580-2.638(t, 2H), 2.236-2.322(m, 7H), 1.865-1.894(d, 2H), 1.677-1.736(d, 2H)

Mass (ESI): m/z238.1/239.1 [m+H]+

Purity by HPLC: NLT 98.0%

Yield: 85-90%

### Step IV

Preparation of N-(3-bromophenyl)-2-methylpropane-2-sulfonamide

Step IV begins with N-(3-bromophenyl)-2-methylpropane-2-sulfinamide.

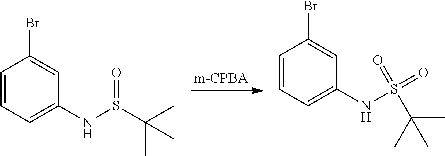

Ethyl acetate (800 mL) and N-(3-bromophenyl)-2-methylpropane-2-sulfinamide (100.0 g, 362.06 mmol) were charged into a round bottom flask at 25° C. and stirred for 15 min. The solution was cooled to 0° C. and mCPBA (97.47 g, 564.8 mmol) was added and stirred at the same temperature for 30 minutes. The temperature was then raised to 25° C. and the mass was stirred for 4 hours. The separated solids were filtered off, the solid (Lot-1) was washed with Ethyl acetate (200 mL). The filtrate was washed with and 5% NaHCO₃ solution (2×600 mL) and the layers were separated. The organic phase was concentrated under reduced pressure. n-Hexane (100 mL) is added and the mixture was stirred for 1 hour at 25° C. The formed solid (Lot-2) was separated by filtration and washed with n-hexane (50 mL). Both the solid lots were combined and slurred in dichloromethane (150 mL) for 1 hour and filtered to get the product as a white solid. The solid was suction dried for 30 minutes and then dried at 55° C. for 6 hours.

The method described as Step IV produced the following results:

H1-NMR Values: 7.440(s, 1H), 7.143-7.241 (m, 3H), 6.355(br, 1H), 1.426(s, 9H)

Mass (ESI): m/z 289.8/291.8 [M-H]+

Purity by HPLC: 98.0%

Yield: 93-95%

### Step V

Preparation of N-(3-(2-chloro-7H-pyrrolo[2,3-d]pyrimidin-7-yl)phenyl)-2-methylpropane-2-sulfonamide

Step V begins with 1,4-Dioxane (1200 mL), N-(3-bromophenyI)-2-methylpropane-2-sulfonamide.

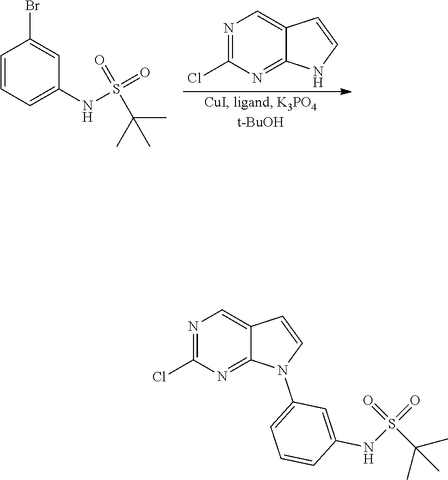 [SMILES: CC(C)(C)S(=O)(=O)Nc1cccc(Br)c1 ; Clc1ncc2cc[nH]c2n1 ; CC(C)(C)S(=O)(=O)Nc1cccc(-n2ccc3cnc(Cl)nc32)c1]

Tert-Butanol (3250 mL), N-(3-bromophenyl)-2-methylpropane-2-sulfonamide (325.0 g, 1.12 mol) and 2-chloro-7H-pyrrolo[2,3-d]pyrimidine (171 g, 1.12 mol) were charged into an autoclave and stirred for 15 minutes at 25° C. Potassium triphosphate (630 g, 2.97 mol), copper iodide (64 g, 0.34 mol) and N, N′-dimethylethylene diamine (59.24 g, 0.67 mol) were added and stirred at 25° C. for 15 minutes. Nitrogen pressure (5 kg/cm2) was applied and the reaction mass temperature was raised to 110° C. The mass was maintained at this temperature for 24 hours. The reaction mass was then cooled to 25-30° C.; diluted with EtOAc (3250 mL.), filtered through hyflow bed. The hyflow bed was washed with EtOAc (1575 mL.). The combined filtrate and washings was concentrated completely under vacuum below 60° C. To the crude material obtained was added 1N HCl (3250 mL.) and EtOAc (3250 mL) at 25-30° C. The reaction mass was stirred for 30 minutes, the solid suspension was filtered on hyflow bed to remove the undissolved solids and layers were separated. The aqueous layer was back extracted with EtOAc (3250 mL) and the combined organic layer was washed with saturated NaHCO₃ solution (3250 mL) followed by water (3250 mL). Concentrated the organic layer completely and the crude material obtained was dissolved in Methanol (675 mL), stirred at 60-65° C. for 0.5-1.0 h. The reaction mass was cooled to 25-30° C., the solid was filtered, washed with methanol (325 mL). The solid obtained was dried under vacuum at 55-60° C. for 10-12 h to get the product as a pale brown colour solid in 36% yield with 98.55% purity by HPLC (specification limit is not less than 98.0%).

The method described as Step V produced the following results:

H1-NMR Values: 9.986(s, 1H), 7.978-7.987(d, 1H), 7.785(s, 1H), 7.466-7.506(t, 1H), 7.371-7.391(d, 1H), 7.292-7.313(d, 1H), 6.912-9.921(d, 1H), 1.337(s, 9H)

Mass (ESI): m/z 364.8/366.9 [M+H]+

Purity by HPLC: NLT 98.0%

Yield: 35-38%

### Step VI

Preparation of N-(3-(2-((4-(4-(dimethylamino)piperidin-1-yl)-3-fluorophenyl)amino)-7H-pyrrolo[2,3-d]pyrimidin-7-yl)phenyl)-2-methylpropane-2-sulfonamide

Step VI combines the structures produced from step III and step V.

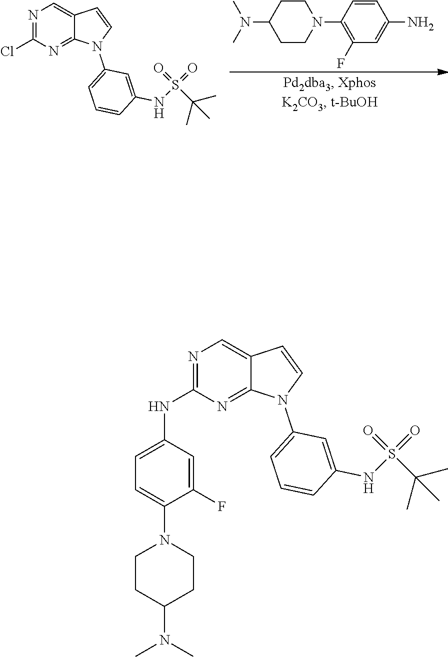 [SMILES: CC(C)(C)S(=O)(=O)Nc1cccc(-n2ccc3cnc(Cl)nc32)c1 ; CN(C)C1CCN(c2ccc(N)cc2F)CC1 ; CN(C)C1CCN(c2ccc(Nc3ncc4ccn(-c5cccc(NS(=O)(=O)C(C)(C)C)c5)c4n3)cc2F)CC1]

To a stirred solution of N-(3-(2-chloro-7H-pyrrolo[2,3-d]pyrimidin-7-yl)phenyl)-2-methylpropane-2-sulfonamide (250 g, 685 mmol) in t-Butanol (2500 mL) in autoclave, were added 1-(4-amino-2-fluorophenyl)-N,N-dimethylpiperidin-4-amine (162.7 g, 685 mmol) K₂CO₃ (236.4 g, 1712 mmol Pd₂(dba)₃ (31.4 g, 34.25 mmol.), X-Phos (32.7 g, 68.5 mmol) sequentially at 25-30° C. The autoclave was closed and the reaction mixture was degassed using N₂ gas for about 15 min. The reaction mixture was heated to 105-110° C. under a Nitrogen pressure of 1-2 Kg/cm² and maintained for about 4-5 h at the same temperature. The reaction was monitored by HPLC for the content of N-(3-(2-chloro-7H-pyrrolo [2, 3-d]pyrim idin-7-yl)phenyl)-2-methylpropane-2-sulfonamide (Limit: NMT 1%). The reaction mass was cooled to 20-30° C. and diluted with 10% Methanol/DCM (2500 mL). Unloaded the reaction mass from autoclave and filtered through hyflow bed. The hyflow bed was washed with 10% Methanol/DCM (1250 mL). Added charcoal (35 g) to the combined filtrate and washings and the reaction mass was heated to 65-70° C. for 1 h. The reaction mass was filtered at the same temperature over hyflow and the hyflow bed was washed with DCM (1000 mL).

The combined filtrate and washings was concentrated to dryness and to the residue was added MeOH (1000 mL) and stirred for 0.5 h at 25-30° C. The solid suspension was filtered, washed with MeOH (1000 mL), and the material was dried under vacuum at 55-60° C. for 5-6 h to get the target base as off white crystalline solid.

The method described as Step VI produced the following results:

X-ray diffraction 2Θ values 8.2, 13.0, 13.5, 16.5, 18.8, 20.1, 20.6+0.2

H1-NMR Values: 9.985(br, 1H), 9.550(s, 1H), 8.795(s, 1H), 7.711-7.768(d, 2H), 7.470- 7.530(m, 4H), 7.282-7.298(d, 1H), 6.949-6.990(t, 1H), 6.671(s, 1H),3.288-3.313(d, 2H), 2.563-2.616(t,2H),2.393(s, 6H),1.900-1.922(d,2H), 1.603-1.627(d, 2H),1.293(s, 9H)

Mass (ESI): m/z 566/566.8 [m+H]+

Purity by HPLC: NLT 98.0%

Yield: 80-82%

### Step VII

Preparation of N-(3-(2((4-(4-(dimethvlamino)piperidin-1-yl)-3-fluorophenyl)amino)-7H-pyrrolo[2,3-d]pyrimidin-7-yl)phenyl)-2-methylpropane-2-sulfonamide salts

Step VII produces a salt of the compound of Formula I.

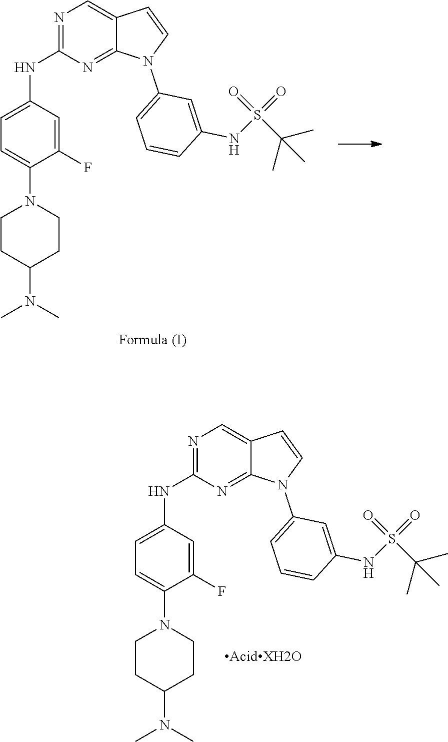 [SMILES: CN(C)C1CCN(c2ccc(Nc3ncc4ccn(-c5cccc(NS(=O)(=O)C(C)(C)C)c5)c4n3)cc2F)CC1 ; CN(C)C1CCN(c2ccc(Nc3ncc4ccn(-c5cccc(NS(=O)(=O)C(C)(C)C)c5)c4n3)cc2F)CC1]

In an alcoholic solvent (Ethanol, Isopropanol, Methanol), N-(3-(2((4-(4-(dimethylamino)piperidin-1-yl)-3-fluorophenyl)amino)-7H-pyrrolo[2,3-d]pyrimidin-7-yl)phenyl)-2-methylpropane-2-sulfonamide base was maintained at room temperature and an equal molar ratio of respective acids in water was added. The mass was stirred at reflux and isolated by filtration at room temperature. The yield was 80-90% with an HPLC purity of greater than 98%. All acid salts taken into alcoholic solvents and isolated pH >8 gave the compound of Formula I. A crystalline form of the Formula I base X-ray diffraction 2Θ values were 8.2, 13.0, 13.5, 16.5, 18.8, 20.1, 20.6+0.2

It will be appreciated that in any of the routes described above, the precise order of the synthetic steps by which the various groups and moieties are introduced into the molecule can be varied. It will be within the skill of the practitioner in the art to ensure that groups or moieties introduced at one stage of the process will not be affected by subsequent transformations and reactions, and to select the order of synthetic steps accordingly. Certain intermediate compounds described above form a yet further aspect of the invention.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present invention, which is defined solely by the claims. Accordingly, the present invention is not limited to that precisely as shown and described.

What is claimed is:

1. A process for preparation of a compound with the structure: 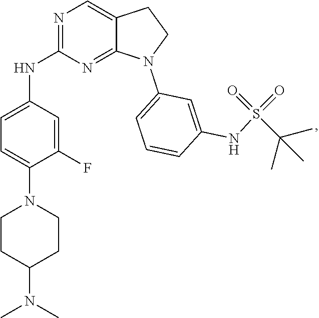 [SMILES: CN(C)C1CCN(c2ccc(Nc3ncc4c(n3)N(c3cccc(NS(=O)(=O)C(C)(C)C)c3)CC4)cc2F)CC1]
a stereoisomer, or a salt thereof, said process comprising steps of:
a) oxidizing a sulfone intermediate with an oxidizing agent in a solvent,
b) condensing the solvent in presence of a metal halide and a catalyst,
c) isolating one or more reaction products from the solvent at one or more pH levels, and
d) identifying a reaction product.

2. The process of claim 1, including a step of treating the reaction product to form a pharmaceutically acceptable acid salt.

3. The process of claim 1, including a step of preparing organic and inorganic acid salts of the reaction product.

4. The process of claim 1, including a step washing and/or rinsing the solution with a base;
wherein the base is selected from carbonates, bicarbonates, hydroxides and organic bases including amines and pyridines, and
wherein the reaction temperature range is −10 to 50° C. and preferably ambient temperature during the washing and/or rinsing steps.

5. The process of claim 1,
wherein the oxidizing agent is KMnO₄, H₂O₂, or m-CPBA,
wherein the solvent is an ester, a toluene, protonated, a ketone, chlorinated, dioxane, DMF, DMAC, other organic solvents, or a C1-C5 alcohols and water, and
wherein the reaction temperature is −10 to 50° C. and preferably ambient temperature.

6. The process of claim 1,
wherein the step of condensing the solvent in presence of a metal halide and a catalyst includes a base,
wherein the base is a carbonate, bicarbonate, hydroxide or organic base such as an amine and pyridine, and
wherein the solvents are preferably esters, toluene, protonated, ketones, dioxanes chlorinated, DMF, DMAC and other organic solvents, C1-C5 alcohols and water,
wherein the reducing agents are 5-10% Pd/C, Zn/NH₄Cl, Fe/HCl,
wherein the reaction pressure is 0 to 10 Kg, and
wherein the reaction temperature is 0 to 100° C., preferably 50-90° C.

7. The process of claim 1,
wherein the step of condensing the solvent in presence of a metal halide and a catalyst includes a solvent that is preferably an ester, toluene, protonated, ketones, dioxanes, chlorinated, DMF, DMAC and other organic solvents, or C1-C5 alcohols and water,
wherein the reaction pressure is 0 to 15 Kg, and
wherein the reaction temperature is 0 to 140° C., preferably less than 90° C.

8. The process of claim 1, including a step washing and/or rinsing the solution with a base,
wherein the base is selected from carbonates, bicarbonates, hydroxides and organic bases such as amines and pyridines,
wherein solvents are preferably esters, toluene, protonated, ketones, dioxanes, chlorinated, DMF, DMAC and other organic solvent, C1-C5 alcohol and water,
wherein the reaction pressure is 0 to 15 Kg,
wherein the reaction temperature is 0 to 140° C., preferably less than 90° C.,
wherein the purification solvents are preferably esters, toluene, protonated, ketones, dioxanes, chlorinated, DMF, DMAC, other organic solvent, or C1-C5 alcohol and water, and
wherein the isolation pH is 0.5-11.0.

9. The process of claim 1, wherein an isolation solvent is used in the step isolating one or more reaction products from the solvent at one or more pH levels,
wherein the isolation solvent is an ester, toluene, protonated, ketone, dioxane, chlorinated, DMF, DMAC or other organic solvent, C1-C5 alcohol, ethyl acetate, methanol, ethanol or isopropanol or chlorinated solvent such as dichloromethane,
wherein the temperature for isolation is 0 to 140° C., preferably greater than 20° C., and
wherein a crystalline form of the reaction product has X-ray diffraction 2Θ values of 8.2, 13.0, 13.5, 16.5, 18.8, 20.1, 20.6±0.2.

10. The process of claim 1, wherein an isolation solvent is used in the step isolating one or more reaction products from the solvent at one or more pH levels,
wherein the isolation solvent is an ester, toluene, protonated, ketone, dioxane, chlorinated, DMF, DMAC or other organic solvent, C1-C5 alcohol, ethyl acetate, methanol, ethanol or isopropanol or chlorinated solvent such as dichloromethane,
wherein the reaction product is a crystalline of a mono, di or tri acid salt with hydrate form,
wherein isolated acid hydrate salts are maleate salts, and
wherein a crystalline form of the reaction product of maleate salt X-ray diffraction has 2Θ values 39.8, 37.6, 36.7, 35.3, 34.9±0.2.

11. The process of claim 1, wherein an isolation solvent is used in the step isolating one or more reaction products from the solvent at one or more pH levels,
wherein the isolation solvent is an ester, toluene, protonated, ketone, dioxane, chlorinated, DMF, DMAC or other organic solvent, C1-C5 alcohol, ethyl acetate, methanol, ethanol or isopropanol or chlorinated solvent such as dichloromethane,
wherein the reaction product is a crystalline of a mono, di or tri acid salt with hydrate form,
wherein isolated acid hydrate salts are succinate salts,
wherein a crystalline form of the reaction product of succinate salt X-ray diffraction has 2Θ values 34.6, 33.1, 31.6, 28.5, 26.4, 25.7, 24.7±0.2.

12. The process of claim 1, wherein an isolation solvent is used in the step isolating one or more reaction products from the solvent at one or more pH levels,
wherein the isolation solvent is an ester, toluene, protonated, ketone, dioxane, chlorinated, DMF, DMAC or other organic solvent, C1-C5 alcohol, ethyl acetate, methanol, ethanol or isopropanol or chlorinated solvent such as dichloromethane,
wherein the reaction product is a crystalline of a mono, di or tri acid salt with hydrate form,
wherein isolated acid hydrate salts are preferably hydrochloride range mono to tri salt hydrates, and
wherein a crystalline form of the reaction product of hydrochloride salt X-ray diffraction has 2Θ values 27.4, 26.1, 24.5, 24.0, 22.6, 20.9, 19.4, 17.9, 10.3±0.2.

13. The process of claim 1, wherein an isolation solvent is used in the step isolating one or more reaction products from the solvent at one or more pH levels,
wherein the isolation solvent is an ester, toluene, protonated, ketone, dioxane, chlorinated, DMF, DMAC or other organic solvent, C1-C5 alcohol, ethyl acetate, methanol, ethanol or isopropanol or chlorinated solvent such as dichloromethane,
wherein the reaction product is a crystalline of a mono, di or tri acid salt with hydrate form,
wherein isolated acid hydrate salts are hydrochloride range mono to tri salt hydrates,
wherein a crystalline form of the reaction product isolated from acid salts X-ray diffraction has 2Θ values of 8.2, 13.0, 13.5, 16.5, 18.8, 20.1, 20.6±0.2.
